(19) United States
(12) Patent Application Publication
(10) Pub. No.: US 2025/0311981 A1
(43) Pub. Date: Oct. 9, 2025

(54) METHODS FOR IDENTIFYING BIOMARKERS PRESENT IN BIOLOGICAL TISSUES, MEDICAL IMAGING SYSTEMS, AND METHODS FOR TRAINING THE MEDICAL IMAGING SYSTEMS

(71) Applicant: OPTINA DIAGNOSTICS, INC., Montreal (CA)

(72) Inventors: Jean-Sebastien GRONDIN, Montreal (CA); Claudia CHEVREFILS, Montreal (CA); Jean-Philippe SYLVESTRE, Montreal (CA)

(21) Appl. No.: 18/962,006

(22) Filed: Nov. 27, 2024

Related U.S. Application Data
(63) Continuation of application No. PCT/IB2023/055423, filed on May 26, 2023.
(60) Provisional application No. 63/346,559, filed on May 27, 2022.

(51) Int. Cl. A61B 5/00; A61B 3/12; G06V 10/56; G06V 10/764; G06V 10/77; G06V 10/776; G06V 10/80; G06V 10/82; G06V 40/18; G16H 10/40; G16H 50/20

(52) U.S. Cl. CPC A61B 5/7267; A61B 3/12; G06V 10/56; G06V 10/764; G06V 10/7715; G06V 10/776; G06V 10/82; G06V 40/193; G16H 10/40; G16H 50/20; G06V 10/806; G06V 2201/031

(57) ABSTRACT

A multispectral imaging system is trained by the application of an unlabelled multispectral image. Artificial labels are added to each image at corresponding wavelengths of the unlabelled multispectral image in view of training a machine learning system. Spatial and then spatial-spectral features of the multispectral images are extracted in successive training phases. The trained machine learning system may then be used to detect biomarkers or other artefacts in a multispectral image by splitting the multispectral image into distinct wavelength-images, applying masks to the wavelength-images to obtain pixel groups, applying statistical calculation to the pixel groups, assembling statistical calculation results into feature vectors, and using the trained machine learning system to extract, from the feature vectors, positive or negative indications related to the presence of biomarkers of other artefacts in the multispectral image. The machine learning system may be retrained upon processing of each subsequent multispectral image.

### CROSS-REFERENCE TO RELATED APPLICATIONS

This application is a continuation of PCT Patent Application No. PCT/IB2023/055423, entitled “METHODS FOR IDENTIFYING BIOMARKERS PRESENT IN BIOLOGICAL TISSUES, MEDICAL IMAGING SYSTEMS, AND METHODS FOR TRAINING THE MEDICAL IMAGING SYSTEMS”, filed May 26, 2023, the entire contents of which are hereby incorporated by reference.

### FIELD

The present technology relates to medical imaging systems and methods. In particular, medical imaging systems are trained and used in view of identifying biomarkers in biological tissues.

### BACKGROUND

Imaging techniques are commonly used to assist in the detection and diagnosis of various illnesses. Images of a particular region of interest (ROI) of a subject are analysed to detect anomalies. A commonly used technique to detect amyloids and other anomalies comprises positron emission tomography (PET) scanning. PET scans are expensive, time consuming, and may cause discomfort to the subject.

One example of anomalies that may be detected and lead to the establishment of a diagnosis comprises amyloids, that is, abnormal protein aggregates. In particular, while Alzheimer's disease is essentially a neurodegenerative illness, it has been shown that the presence of manifestations, in the retina of a subject, of the presence of amyloid plaques may indicate the onset of Alzheimer's disease. It has further been proposed to the diagnosis of other illnesses, for example diabetes and cardiovascular diseases including hypertension, could be based on the detection of anomalies within the retina of a subject.

Recently, techniques involving numerical image processing have been proposed. In particular, image analysis may be used to characterize image textures with the aim of discovery abnormal patterns within a ROI of the subject. Currently, there are few commercially available technologies capable of efficiently detecting, within the eye of a subject, a manifestation of a disease rooted in other organs of the subject. An example of such technology may be found in International Patent Application Publication No. WO 2016/041062 to Sylvestre et al., the disclosure of which is incorporated by reference herein in its entirety, which discloses techniques for producing spectrally resolved images that may be used for identifying retinal features that correlate with presence of amyloid in the brain of a subject suffering from the onset of Alzheimer's disease.

FIG. 1 (Prior Art) is a block diagram of an apparatus for producing a spectrally resolved image of a retina of a subject, the apparatus being introduced in WO 2016/041062. An apparatus 100 can produce a hyperspectral image of the complete retina or of any part thereof, for example an image of the optical nerve. The apparatus 100 comprises a light source 110, a tunable filter 120, an illuminating optic component 130, a collecting optic component 140, a blocking filter 150, a sensor 160, a processor 170 and a display 180. The light source 110 as shown produces light having a broad wavelength range, for example white light. The wavelength range of the light source 110 may further extend in the ultraviolet and/or infrared ranges. The tunable filter 120 has a high out-of-band rejection and extracts monochromatic excitation light 122 from the light source 110. The illuminating optic component 130 may comprise one or more lenses, one or more optic fibers, or an assembly thereof. It directs the monochromatic excitation light 122 towards the retina 190 of the subject. The illuminating optic component 130 may illuminate at once the entire retina 190 or a section of the retina 190 under the control of an operator of the apparatus 100. Alternatively, the optic component 130 may comprise a scanning apparatus (not shown) effecting a raster scan of the retina 190 by directing the monochromatic excitation light 122 to image the retina 190 one pixel at a time. The collecting optic component 140 may comprise one or more lenses, one or more optic fiber, or an assembly thereof. It collects light emanating from the retina 190 of the subject. This light includes a fraction 142 of the monochromatic excitation light 122 and an additional fluorescence signal 144. The blocking filter 150 blocks, separates or removes the fraction 142 of monochromatic excitation light 122 from the fluorescence signal 144 emanating from the retina 190 of the subject. The blocking filter 150 attenuates wavelengths in a range of the excitation light 122 while passing with minimal attenuation wavelengths of the fluorescence signal 144. The sensor 160 senses the filtered fluorescence signal 144. The processor 170 controls the tunable filter 120 to iteratively select wavelengths of the monochromatic excitation light 122. The processor 170 may cause the tunable filter 120 to output the monochromatic excitation light 122 by sweeping over a range extending from 350 to 1000 nm, or over a part of this range. The processor 170 produces the spectrally resolved image of the retina 190 based on the fluorescence signal that emanates from the retina 190 of the subject. The display 180, if present, shows the spectrally resolved image of the retina 180. In an example, of the apparatus 100, the tunable filter 120 may attenuate out-of-band emission of the monochromatic excitation light 122 by a factor of at least 10,000 to 1 (OD 4) at 20 nm from the nominal wavelength.

The light emanating from the retina 190 may comprise light 142 reflected by the retina 190 or a fluorescence signal 144 emitted by the retina, the reflected light or the fluorescence signal resulting from directing the monochromatic excitation light 122 towards the retina 190 of the subject.

The sensor 160 may comprise a camera capable of capturing light in spectral ranges of the reflected light and of the fluorescence signal. The light source 110 may comprise a broadband light source, for example a supercontinuum light source, the tunable filter 120 may comprise a volume Bragg grating filter or other type of filter having high out-of-band rejection, and/or the blocking filter 150 may comprise a tunable blocking filter or a plurality of blocking filters, for example mounted on a filter wheel, and be configured to allow passing of the fluorescence signal 144 in a plurality of wavelengths, allowing fluorescence imaging in multiple spectral ranges.

The light source 110 may alternatively comprise a tunable light source emitting monochromatic light with high out-of-band rejection, the light source 110 having an OD of at least 4.0 or up to 4.7.

The tunable filter 120 may output the monochromatic excitation light in a 350 to 1000 nm wavelength range, tunable in 0.1 to 10 nm increments. The blocking filter 150 may be a bandpass filter having a bandwidth in a 20 to 100 nm range.

In addition to the above mentioned functions, the processor 170 analyzes the spectral image of the retina 190. This type of analysis allows to identify spectral signatures within the spectrally resolved image of the retina 190, to identify location and concentration of biomarkers on the spectrally resolved image of the retina 190, to normalize the spectrally resolved image of the retina 190, to correct the spectrally resolved image of the retina 190 according to spectral characteristics of the apparatus 100 and its optical components, to perform registration of the spectrally resolved image of the retina 190 to correct for eye movements of the subject, or to perform any combination of these functions.

FIG. 2 (prior art) is a representation of regions of interest of two subjects, one of which being amyloid positive. In FIG. 2, the ROI is within the retina of the two subjects. Photograph 10_{A} shows a ROI for an amyloid positive subject while photograph 10_{B} shows a similar ROI for an amyloid negative subject. While photographs 10_{A} and 10_{B} do reveal some differences between these ROIs, these differences are subtle and may not suffice to easily discriminate between normal and abnormal conditions. Diagnosis based on photographs 10_{A} and 10_{B} requires the attention of a highly skilled medical professional. In spite of the skill of the medical professional, diagnosis errors may occur due to the ambiguous distinction between photographs 10_{A} and 10_{B} that respectively show abnormal and normal tissues.

Image texture analysis has been proposed as a tool for representing ROIs while highlighting evidence of potential anomalies. An example of a numerical image processing technique using image texture analysis may be found in United States Patent Application Publication No. 10,964,036 to Sylvestre et al., the disclosure of which is incorporated by reference herein in its entirety. This disclosure introduces techniques for producing spectrally resolved images that may be used for identifying retinal features that correlate with presence of amyloid in the brain of a subject suffering from the onset of Alzheimer's disease.

FIG. 3 (Prior Art) is a schematic representation of a process for using a moving window to build a texture image of a biological tissue based on spatial and spectral information, the process being disclosed in U.S. Pat. No. 10,964,036. On FIG. 3, the biological tissue is found in a region of interest (ROI) of a subject. An organ or tissue 50 of a subject contains a ROI 52 from which a plurality of images 54₁, 54₂ . . . 54ⱼ are obtained at j distinct wavelengths to generate a hyperspectral image 56 of the ROI 52. Each one of the plurality of images 54₁, 54₂ . . . 54ⱼ may be obtained by capturing reflectance or fluorescence emitted from the ROI 52. The images 54₁, 54₂ . . . 54ⱼ as well as the hyperspectral image 56 each contain a plurality of pixel rows 58 and a plurality of pixel columns 60. A portion of the hyperspectral image 56, in a window 62, contains spatial information over a width of k pixels and a height of l pixels, in which each of k and l are greater than or equal to one (1) pixel, this window 62 also containing spectral information 64 defined over the j distinct wavelengths. A texture analysis of the hyperspectral image 56 is performed based on spatial information contained in the k·l pixels of the window 62, the texture analysis being resolved over the j distinct wavelengths. By moving the window 62 over the area of the ROI 52, the texture analysis provides a texture image 20_{B} of the ROI 52. The texture image 20_{B} contains information describing the ROI 52, for example a normalised contrast image, a normalised homogeneity image, a normalised correlation image and/or a normalised energy image of the ROI 52.

The above-described technologies facilitate the visualisation of defects in a biological tissue and the classification of the biological tissue as normal or abnormal. There may remain a need to rely on the expertise of a seasoned medical practitioner to identify a biomarker in the biological tissue, particularly when various abnormal biomarkers may be present in the biological tissue. Some imaging systems may be trained to recognize abnormal biomarkers. However, such training requires the provision of very large numbers of images, for examples thousands of images having assorted labels identifying the known presence of such biomarkers. Collecting and labelling these images is a serious problem, as it is very time consuming. Considering that labelling of these images may only be performed by experienced medical practitioners, training imaging systems may be prohibitively expensive. Also, providing thousands of labelled images for training causes a serious processing burden on the imaging systems. The requirement to provide very large numbers of labelled images for training imaging systems has so far seriously limited technological developments.

Therefore, even though the recent developments identified above may provide benefits, improvements are still desirable.

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches.

### SUMMARY

Embodiments of the present technology have been developed based on developers' appreciation of shortcomings associated with the prior art.

In particular, such shortcomings may comprise the need to rely on the expertise of a seasoned medical practitioner to identify a biomarker in an image of a biological tissue, particularly when various abnormal biomarkers may be present in the biological tissue, and the serious processing burden related to the training of medical imaging systems.

In the context of the present specification, unless expressly provided otherwise, a computer system may refer, but is not limited to, an “electronic device”, an “operating system”, a “system”, a “computer-based system”, a “controller unit”, a “monitoring device”, a “control device”, an “artificial intelligent system” supporting machine learning features including (or not) deep learning features, and/or any combination thereof appropriate to the relevant task at hand.

In the context of the present specification, unless expressly provided otherwise, the expression “computer-readable medium” and “memory” are intended to include media of any nature and kind whatsoever, non-limiting examples of which include RAM, ROM, disks (CD-ROMs, DVDs, floppy disks, hard disk drives, etc.), USB keys, flash memory cards, solid state-drives, and tape drives. Still in the context of the present specification, “a” computer-readable medium and “the” computer-readable medium should not be construed as being the same computer-readable medium. To the contrary, and whenever appropriate, “a” computer-readable medium and “the” computer-readable medium may also be construed as a first computer-readable medium and a second computer-readable medium.

In the context of the present specification, unless expressly provided otherwise, the words “first”, “second”, “third”, etc. have been used as adjectives only for the purpose of allowing for distinction between the nouns that they modify from one another, and not for the purpose of describing any particular relationship between those nouns.

Implementations of the present technology each have at least one of the above-mentioned object and/or aspects, but do not necessarily have all of them. It should be understood that some aspects of the present technology that have resulted from attempting to attain the above-mentioned object may not satisfy this object and/or may satisfy other objects not specifically recited herein.

Additional and/or alternative features, aspects and advantages of implementations of the present technology will become apparent from the following description, the accompanying drawings and the appended claims.

### BRIEF DESCRIPTION OF THE DRAWINGS

For a better understanding of the present technology, as well as other aspects and further features thereof, reference is made to the following description which is to be used in conjunction with the accompanying drawings, where:

FIG. 1 (Prior Art) is a block diagram of an apparatus for producing a spectrally resolved image of a retina of a subject;

FIG. 2 (Prior Art) is a representation of regions of interest of two subjects, one of which being amyloid positive;

FIG. 3 (Prior Art) is a schematic representation of a process for using a moving window to build a texture image of a biological tissue based on spatial and spectral information;

FIG. 4 is a block diagram of a medical imaging system in accordance with an embodiment of the present technology;

FIG. 5 is an illustration of a method for detecting biomarkers in a biological tissue in accordance with an embodiment of the present technology;

FIGS. 6a and 6b are a sequence diagram showing operations of the method for detecting biomarkers in a biological tissue in accordance with an embodiment of the present technology;

FIG. 7 is an illustration of a first pretext task for training a medical imaging system in accordance with an embodiment of the present technology;

FIG. 8 is an illustration of a second pretext task for training a medical imaging system in accordance with an embodiment of the present technology;

FIG. 9 is an illustration of a target task for training a medical imaging system in accordance with an embodiment of the present technology;

FIGS. 10a to 10d are a sequence diagram showing operations of a first pretext task for training a medical imaging system in accordance with an embodiment of the present technology;

FIGS. 11a to 11d are a sequence diagram showing operations of the second pretext task for training a medical imaging system in accordance with an embodiment of the present technology;

FIGS. 12a and 12b are a sequence diagram showing operations of the target task for training a medical imaging system in accordance with an embodiment of the present technology; and

FIG. 13 is a block diagram of a processing system part of the medical imaging system in accordance with an embodiment of the present technology.

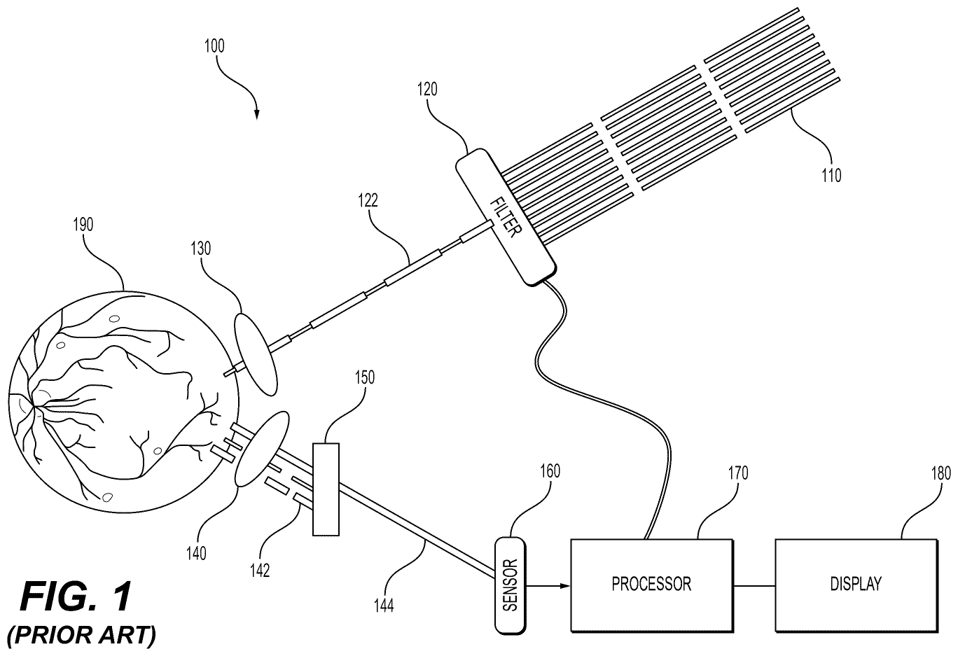

It should also be noted that, unless otherwise explicitly specified herein, the drawings are not to scale.

### DETAILED DESCRIPTION

The examples and conditional language recited herein are principally intended to aid the reader in understanding the principles of the present technology and not to limit its scope to such specifically recited examples and conditions. It will be appreciated that those skilled in the art may devise various arrangements that, although not explicitly described or shown herein, nonetheless embody the principles of the present technology.

Furthermore, as an aid to understanding, the following description may describe relatively simplified implementations of the present technology. As persons skilled in the art would understand, various implementations of the present technology may be of a greater complexity.

In some cases, what are believed to be helpful examples of modifications to the present technology may also be set forth. This is done merely as an aid to understanding, and, again, not to define the scope or set forth the bounds of the present technology. These modifications are not an exhaustive list, and a person skilled in the art may make other modifications while nonetheless remaining within the scope of the present technology. Further, where no examples of modifications have been set forth, it should not be interpreted that no modifications are possible and/or that what is described is the sole manner of implementing that element of the present technology.

Moreover, all statements herein reciting principles, aspects, and implementations of the present technology, as well as specific examples thereof, are intended to encompass both structural and functional equivalents thereof, whether they are currently known or developed in the future. Thus, for example, it will be appreciated by those skilled in the art that any block diagrams herein represent conceptual views of illustrative circuitry embodying the principles of the present technology. Similarly, it will be appreciated that any flowcharts, flow diagrams, state transition diagrams, pseudo-code, and the like represent various processes that may be substantially represented in non-transitory computer-readable media and so executed by a computer or processor, whether or not such computer or processor is explicitly shown.

The functions of the various elements shown in the figures, including any functional block labeled as a “processor”, may be provided through the use of dedicated hardware as well as hardware capable of executing software in association with appropriate software. When provided by a processor, the functions may be provided by a single dedicated processor, by a single shared processor, or by a plurality of individual processors, some of which may be shared. In some embodiments of the present technology, the processor may be a general-purpose processor, such as a central processing unit (CPU) or a processor dedicated to a specific purpose, such as a digital signal processor (DSP), or a neural network comprising a plurality of neurons in one or more layers. Moreover, explicit use of the term a “processor” should not be construed to refer exclusively to hardware capable of executing software, and may implicitly include, without limitation, application specific integrated circuit (ASIC), field programmable gate array (FPGA), read-only memory (ROM) for storing software, random access memory (RAM), and non-volatile storage. Other hardware, conventional and/or custom, may also be included.

Software modules, or simply modules which are implied to be software, may be represented herein as any combination of flowchart elements or other elements indicating performance of process operations and/or textual description. Such modules may be executed by hardware that is expressly or implicitly shown. Moreover, it should be understood that module may include for example, but without being limitative, computer program logic, computer program instructions, software, stack, firmware, hardware circuitry or a combination thereof which provides the required capabilities.

In a first aspect of the present technology, one or more biomarkers present in a biological tissue may be identified by considering M images of the biological tissue. Masks are applied to each of the M images, each image having been captured at one of M respective wavelengths. Statistical calculations are applied to each of the masked images to produce feature vectors. A probability that a given biomarker is present in the biological tissue is extracted from the feature vectors. A trained transformer encoder is used to output class embeddings and a trained classification head is used to output, from the class embeddings, the probability that the given biomarker is present in the biological tissue. The transformer encoder and the classification head may be trained using a plurality of distinct biological tissues and may be trained further following the consideration of the further groups of biological tissue images.

In a second aspect of the present technology, a medical imaging system is trained using M images of a biological tissue, each of the M images containing light at one of M respective wavelengths, the M images collectively forming a multispectral image of the biological tissue. N images are selected among the M images of the biological tissue and one or more artificial labels are added to each of the N selected images. A convolutional model is trained by applying thereto the N selected images including the artificial labels. The convolutional model outputs a first feature vector for each of the N selected images. One or more classification heads are trained by inputting, to each of the one or more classification heads, a combination (for example a concatenation) of the M first feature vectors outputted by the convolutional model to cause the one or more classification heads to identify the one or more artificial labels. Once trained, the transformer encoder and the one or more classification heads may be used to identify one or more biomarkers present in biological tissues. Using artificial labels for training the medical imaging system reduces the burden of medical practitioners who do not need to provide actual labels for the multispectral image containing the M images and reduces the processing burden in training the medical imaging system.

It may be noted that the above-mentioned first aspect of the present technology, which relates to the identification of one or more biomarkers present in a biological tissue, may build on top of the present second aspect of the present technology, which describes training of a medical imaging system. It should be noted, however, that other training techniques may be used in relation to the first aspect of the present technology.

With these fundamentals in place, we will now consider some non-limiting elements to illustrate various implementations of aspects of the present technology.

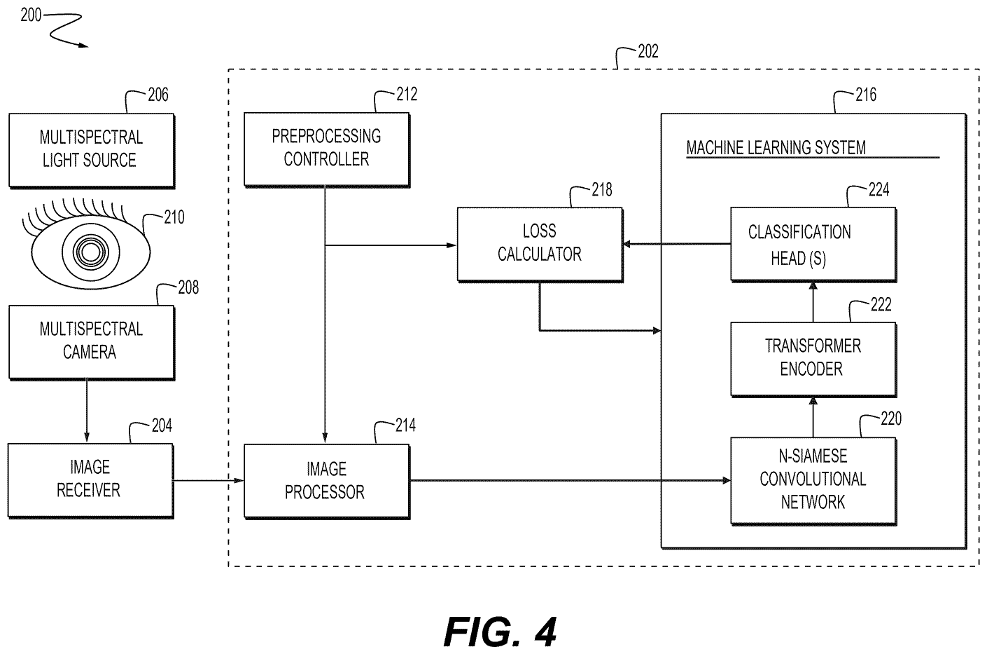

FIG. 4 is a block diagram of a medical imaging system 200. The medical imaging system 200 comprises a processing system 202, an image receiver 204 and may comprise an image acquisition unit including a multispectral light source 206 and a multispectral camera 208. The multispectral light source 206 and the multispectral camera 208, both of which being are positioned in view of a biological tissue 210 for capturing an image thereof. The biological tissue 210 may for example be the retina of an eye of a subject, or a portion of the skin of a subject. Acquiring an image using a laparoscopic imaging system or any suitable imaging probe is also contemplated.

The multispectral light source 206 may emit white light including all or a substantial portion of the visible spectrum. Alternatively, the multispectral light source 206 may emit light over a broader spectrum, including for example some of the infrared spectrum. The multispectral light source 206 may for example be a hyperspectral light source. Similarly, the multispectral camera 208 may be configured to acquire light reflected by the biological tissue 210 over a plurality of spectral bands, being for example a standard RGB (red-green-blue) camera, a camera having an extended spectral capability, or a hyperspectral camera.

In some embodiments, the image acquisition unit may be distinct and separate from the medical imaging system 200, in which case a multispectral image of the biological tissue 210 may be received at the image receiver 204 from any source including, for example and without limitation, from a network via a communication link, from a computer disk, from a portable memory device, and the like.

The processing system 202 comprises a plurality of modules that may be implemented using a plurality of cooperating processors. These modules include a preprocessing controller 212, an image processor 214, a machine learning system 216 and a loss calculator 218. The machine learning system 216 may be implemented using a neural network or any other suitable artificial intelligence technology. In a non-limiting embodiment, the machine learning system 216 may comprise a N-Siamese convolutional network 220, a transformer encoder 222 and one or more classification heads 224.

Generally speaking, the image processor 214 splits the multispectral image obtained by the image receiver 204 into multiple images at various wavelengths and manipulates each resulting image according to instructions received from the preprocessing controller 212. The thus manipulated images are applied to the machine learning system 216 for processing.

During training of the machine learning system 216, a result of the processing performed by the machine learning system 216 may be compared with the instructions from the preprocessing controller 212 by the loss calculator 218. A resulting loss value may be used to train and incrementally update the parameters of the machine learning system 216 in making predictions about the presence of artefacts in the multispectral images. After training, in inference mode, the medical imaging system 200 may be used to predict with enhanced accuracy the presence of biomarkers in multispectral images. In an aspect, the trained machine learning system 216 may be able to predict the presence of biomarkers that are not visible to the naked eye in the multispectral images acquired by the image acquisition unit.

Many variants of the general architecture of the medical imaging system 200 will be described hereinbelow.

### Multispectral Image Feature Extraction Using Anatomical Masks

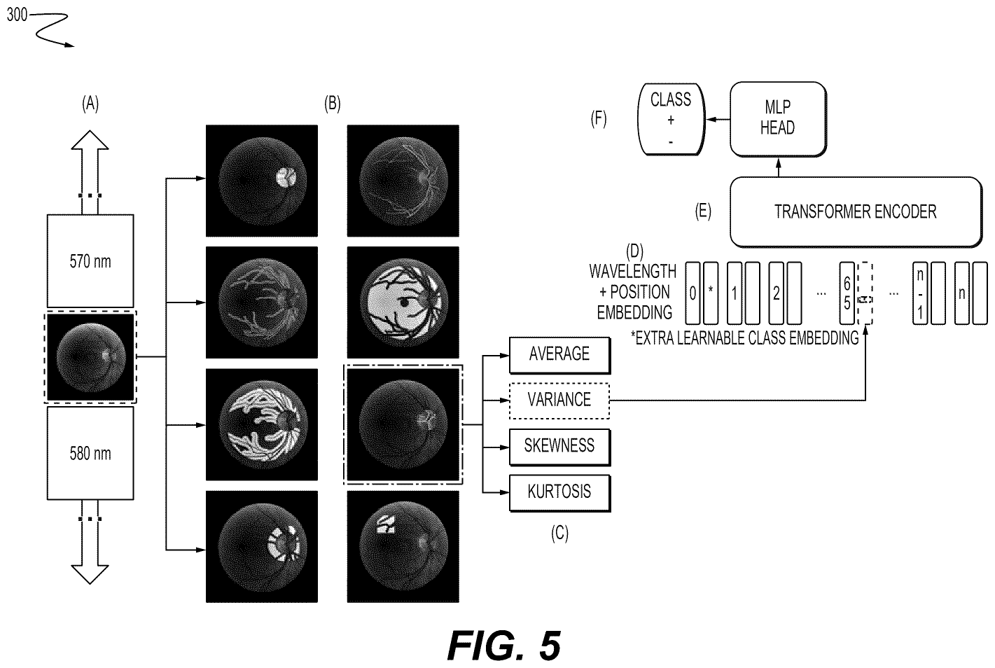

FIG. 5 is an illustration of a method 300 for detecting biomarkers in a biological tissue. This method 300 introduces a multispectral image (for example an RGB image or a hyperspectral image) feature extraction technique by applying anatomical masks to images of a biological tissue obtained at multiple wavelengths. This feature extraction technique greatly increases the capabilities of a transformer encoder and of a classification head for the provision of indications related to the presence of biomarkers in the biological tissue.

In one aspect, a hyperspectral image of the retina of a subject is decomposed into images at N wavelengths and relevant spatial-spectral features that characterize anatomical regions at the different wavelengths are extracted for subsequent input in a transformer encoder.

In more details, the hyperspectral image is split into multiple wavelengths (A) and pixel values from each wavelength are subsampled using a variety of anatomical masks to obtain different groups of pixel values (B). The anatomical masks used in the feature extraction are selected in light of their discriminatory power for a given classification task, for example for classifying the presence of specific biomarkers. Some examples of anatomical masks applicable to retinal images include arteries, veins, the optic nerve head without vessels, the vessels inside the optic nerve head, the vessels neighboring pixels, the retinal background, and the like. Other types of anatomical masks may be used when evaluating, for example, an image of skin or an image obtained via laparoscopy.

Relevant statistics, for example an average, a variance, a skewness and/or a kurtosis are computed for each group of pixel values (C). Hence, for each wavelength, a feature vector is obtained with a size equal to the total number of anatomical masks and statistics pairs. For example, with 8 anatomical region masks and 4 statistic types, a feature vector of size 32 is obtained for each wavelength. For each wavelength, the feature vector is summed with a positional encoding specific to the wavelength position in the sequence shown in (A) to form a sequence of spectral embeddings (D).

The spatial-spectral feature vectors are then used as input to a transformer encoder (E). Transformer encoders are designed to treat sequential inputs and, in the present technology, the transformer encoder can use the positional information obtained in (D) as an indication of the sequence.

The transformer encoder outputs an embedding of a chosen size M (for example 128) for each step of the sequence. At least one of these embeddings is applied to a classification head, which may for example be implemented as a multi-layer perceptron composed of 0, 1 or 2 fully connected hidden layers and 1 fully connected classification layer. The classification head takes as input the class embedding from the transformer encoder and outputs a positive or negative indication for the presence of a certain biomarker in the retina of the subject.

When the method 300 is used with a trained machine learning system, unlabelled images may be applied and the classification head provides the desired indication about the eventual presence of a biomarker in the retina (or other biological tissue) of the subject. Labelled images are used in the training phase. The application of well-chosen anatomical masks to the images at each wavelength and the statistical calculations applied to the masked images is expected to allow to train the machine learning system with a much smaller number of labelled images when compared to conventional training techniques.

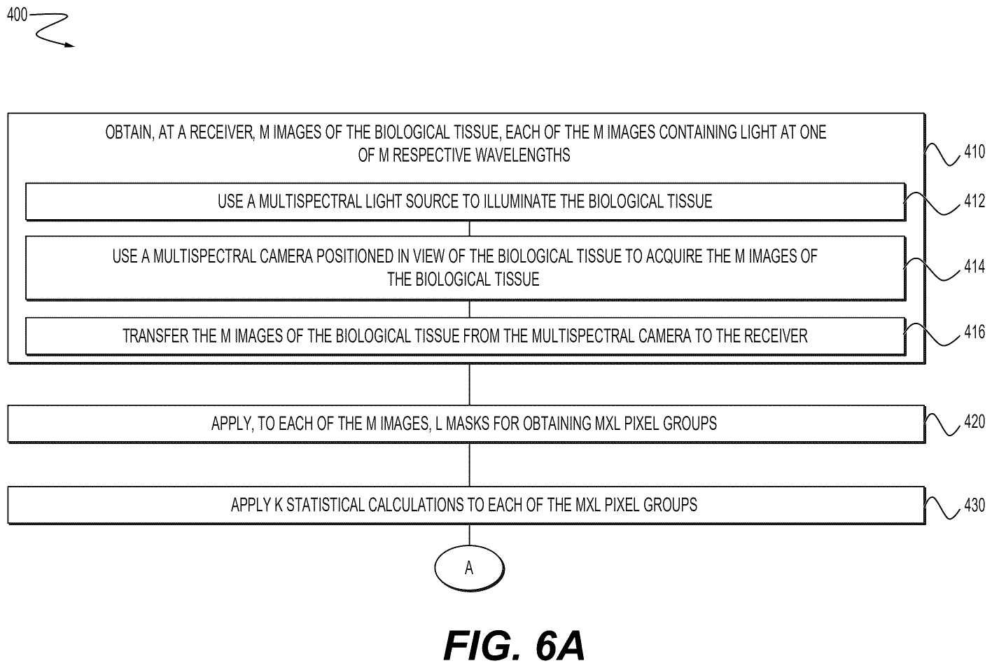

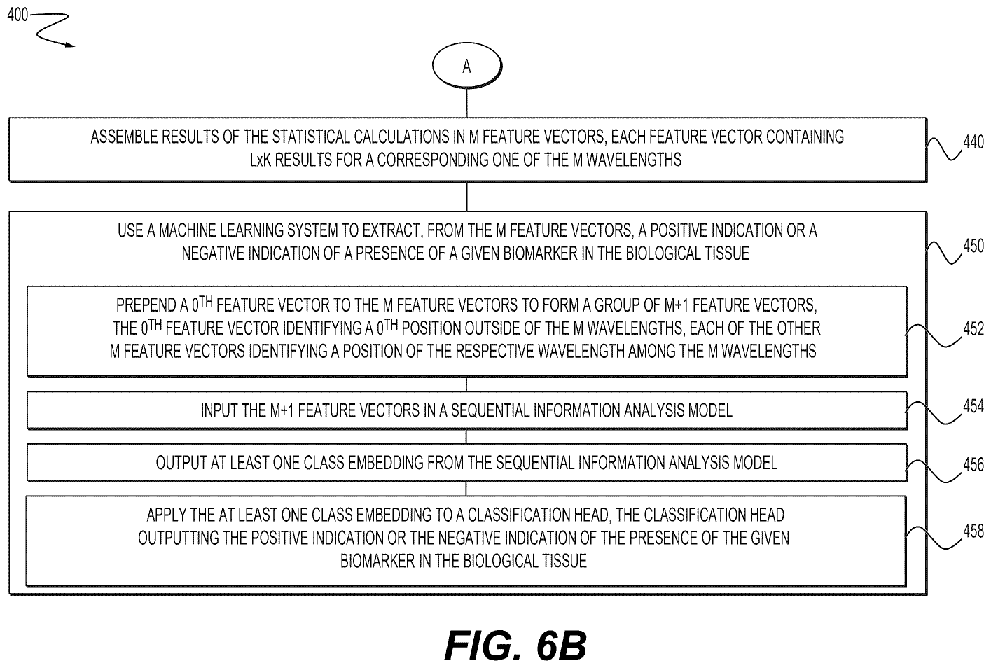

FIGS. 6a and 6b are a sequence diagram showing operations of the method for detecting biomarkers in a biological tissue. The biological tissue may, for example and without limitation, be a retina of a subject, a portion of the skin of a subject, or any other anatomical image containing light at two or wavelengths. On FIGS. 6a and 6b, a sequence 400 comprises a plurality of operations, some of which may be executed in variable order, some of the operations possibly being executed concurrently, some of the operations being optional.

At operation 410, a receiver obtains M images of the biological tissue, each of the M images containing light at one of M respective wavelengths. In a non-limiting embodiment, M may include 91 distinct wavelengths. Operation 410 may comprise one or more sub-operations 412, 414 and 416. At sub-operation 412, a multispectral light source is used to illuminate the biological tissue. At sub-operation 414, a multispectral camera positioned in view of the biological tissue is sued to acquire the M images of the biological tissue. The M images of the biological tissue are transferred from the multispectral camera to the receiver at sub-operation 416. Alternatively, the receiver may obtain the M images of the biological tissue from a network via a communication link, from a computer disk, from a portable memory device, and the like.

One or more masks are applied to each of the M images, at operation 420. In a non-limiting embodiment, 8 distinct masks may be applied. These mask are used as samplers selecting portions of each of the M images to form M pixel groups for each mask. In this context, each of the one or more masks may be an anatomical mask configured to highlight, in the M images of the biological tissue, a corresponding pixel group defining, for example, an optic nerve hypoplasia, a blood vessel, an optic nerve head, a vessel inside the optic nerve head, a contour of a blood vessel, pigment spots, a drusen, or a retinal background.

One or more statistical calculations are applied to each of the pixel groups at operation 430. In a non-limiting embodiment, 4 distinct statistical calculations may be applied. The one or more statistical calculation may comprise, for example and without limitation, an average, a variance, a skewness, a kurtosis, a standard deviation, a median, a smallest value, a largest value, a first, second or third quartile, and any combination thereof. Each combination of a mask with a statistical calculation has a particular discriminatory power for classifying a presence of a specific biomarker in the biological tissue.

At operation 440, the results of the statistical calculations are assembled in one feature vector for each of the M images (i.e., for each of the M wavelengths). These M feature vectors may be called spatial-spectral feature vectors because they contain both spatial and spectral information. Each feature vector containing for a corresponding one of the M wavelength a number of results equal to a number of the masks times a number of the statistical calculations.

Then at operation 450, a machine learning system, which may or may not have been trained earlier, is used to extract, from the M feature vectors, a positive indication or a negative indication of a presence of a given biomarker in the biological tissue.

Operation 450 may comprise one or more sub-operations 452, 454, 456 and 458. At sub-operation 452, a 0^{th} feature vector is prepended to the M feature vectors to form a group of M+1 feature vectors, the 0^{th} feature vector identifying a 0^{th} position outside of the M wavelengths, each of the other M feature vectors identifying a position of the respective wavelength among the M wavelengths. The 0^{th} feature vector may be randomly initialized so that it doesn't contain any useful information on its own. The M+1 feature vectors are input in a sequential information analysis model at sub-operation 454. The sequential information analysis model may be formed of a neural network and may comprise, for example and without limitation, a transformer encoder, a long short-term memory model and a recurrent neural network. At sub-operation 456, at least one class embedding is output from the sequential information analysis model. Then at operation 458, the at least one class embedding is applied to a classification head that, in turn, outputs the positive or negative indication of the presence of the given biomarker in the biological tissue. In a non-limiting embodiment, the at least one class embedding applied to the classification head is a 0^{th} class embedding of M+1 class embeddings outputted from the sequential information analysis model.

The sequence 400 may be used for training, or for retraining, the machine learning system used in operation 450. To this end, the sequence 400 may be applied to a plurality of groups of images, each group of images corresponding to one of a plurality of biological tissues, each group of images including M respective images containing light the M respective wavelengths. A respective model loss may be calculated for each group of images by comparing a respective label contained in the group of images with a respective positive or negative indication of the presence of the given biomarker in the corresponding biological tissue. The calculated model loss may be used to train or retrain the machine learning system.

### Self-Supervised Learning Framework for Multispectral Image

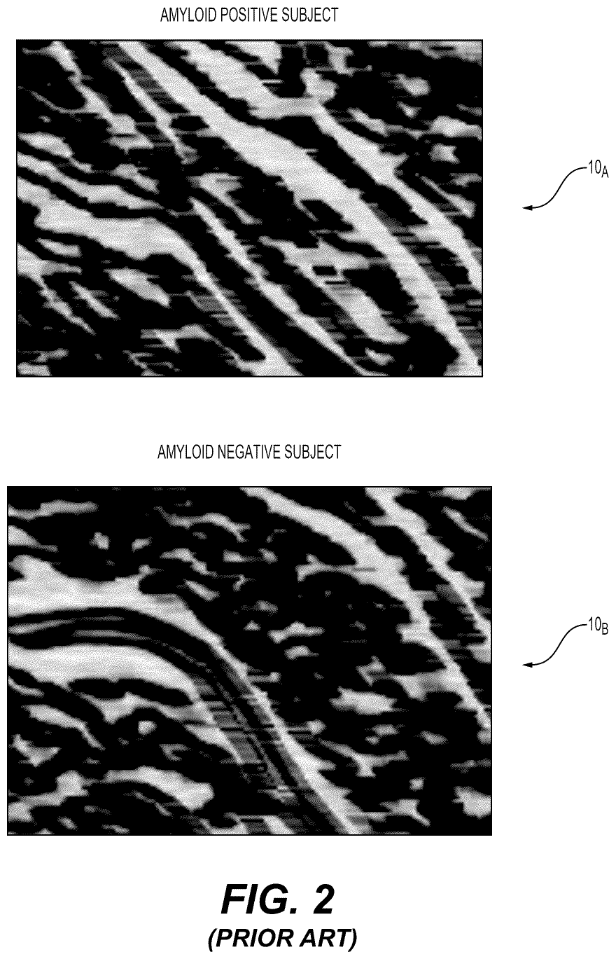

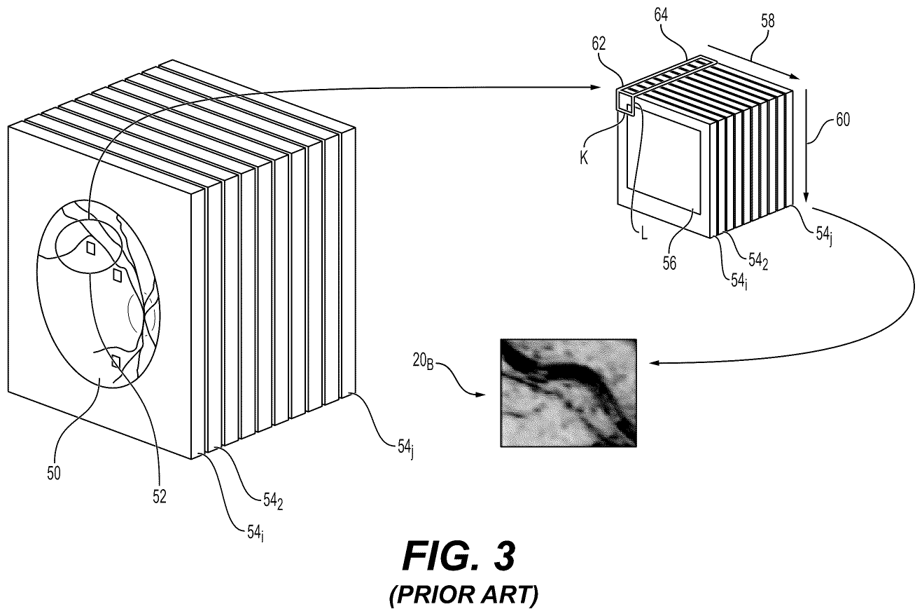

In an embodiment of the medical imaging system, the image receiver 204 (FIG. 2) obtains the multispectral image (M images at M corresponding wavelengths), for example from a multispectral camera 208 such as a color (RGB) camera or a hyperspectral camera. The M images are submitted to the image processor 214, which is configured to selectively apply various transformations to the M images. These transformations may include for example grouping of the M images into N groups, shuffling the N groups, selecting one image in each of the N groups or calculating a texture image based on each of the N groups, selectively applying a geometric transformation to some of the images, and selectively applying a mask on the images. The preprocessing controller 212 provides instructions to the image processor 214 for controlling these various image transformations. The thus modified images are applied to the machine learning system 216 that includes the N-Siamese convolutional network 220, the transformer encoder 222 and at least one classification head 224. The classes obtained by the machine learning system 216 are applied to the loss calculator 218 that compares the resulting classes to the instructions that have been used to control the image transformations. Differences (e.g., losses) between the actual image transformations and the resulting classes are fed back to the machine learning system 216. In the machine learning system 216, weights and parameters are adjusted based on the fed back differences. In this manner, the machine learning system 216 is trained by the application of a number of multispectral images, which are treated until the machine learning system 216 learns to minimize classification errors. Training is performed over a first pretext task, a second pretext task and then a target task.

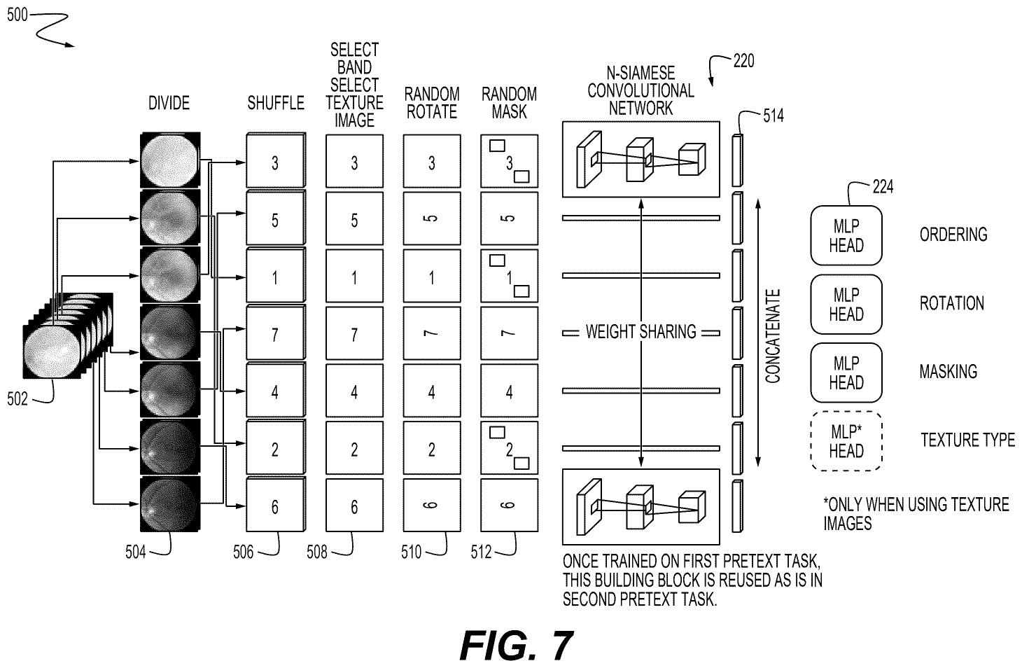

FIG. 7 is an illustration of a first pretext task 500 for training a medical imaging system. In the first pretext task, self-supervised learning is used to construct a spatial feature extractor by taking an image as input and outputting a series of vectors, each vector including characteristic information regarding spatial information of the multispectral image at each wavelength, in the form of parameters that are relevant for the classification task. A neural network able to process these parameters may be used as a platform for building a machine learning system. The spatial feature extractor is constructed without any a priori knowledge of the presence or absence of a biomarker in a particular image. the medical imaging system thus learns about the anatomy of the biological tissue in a global manner and learns what should be visible at each given wavelength. This allows to pretrain the model with a relatively small number of images with good performance. This is economical because it does not require training with a vast number of labelled images.

To this end, preprocessing is used to alter an unlabelled multispectral image 502 including M wavelengths to introduce a number of artificial labels related to image modifications. This preprocessing is performed upstream of the machine learning system 216. The multispectral image 502 may be split into N groups 504 of images, each image containing light at one of the M wavelengths. As a non-limiting example, the multispectral image 502 may include 91 wavelengths and 7 groups 504 may be formed, each group 504 containing 13 images. In an embodiment, various numbers of images may be placed in each group 504. The positions of the groups 504 may then be shuffled, either randomly or according to a predetermined sequence, forming N shuffled groups 506. One image 508 is selected from each group. The N selected images 508 may be randomly selected among the N shuffled groups 506. Alternatively, each selected image 508 may be a texture image calculated on the basis of the images contained in each shuffled group 506. A geometric transformation, for example a rotation, may selectively be applied to one of more of the N first selected images 508, producing N selected (and transformed) images 510. Finally, a mask may be selectively applied to the N selected images 510, producing N selected (and masked) images 512. The mask may be selected among a plurality of anatomical masks. Application of various random masks, for example and without limitation a square or any other shape, is also contemplated.

The N selected (and masked) images 512 are applied for training a convolutional model, for example the N-Siamese convolutional network 220, or another neural network, that outputs a spatial feature vector 514. The spatial feature vector 514 is then applied to the one or more classification heads 224 that output positive or negative indications of the various artificial labels. As expressed in the discussion of FIG. 2, these indications may be compared with the actually applied artificial labels in order to calculate losses and update parameters of the machine learning system 216. In this first pretext task 500, the classification heads may be specially configured to identity the order, geometric transformation (e.g., rotation), masking and texture type for the N selected images 512.

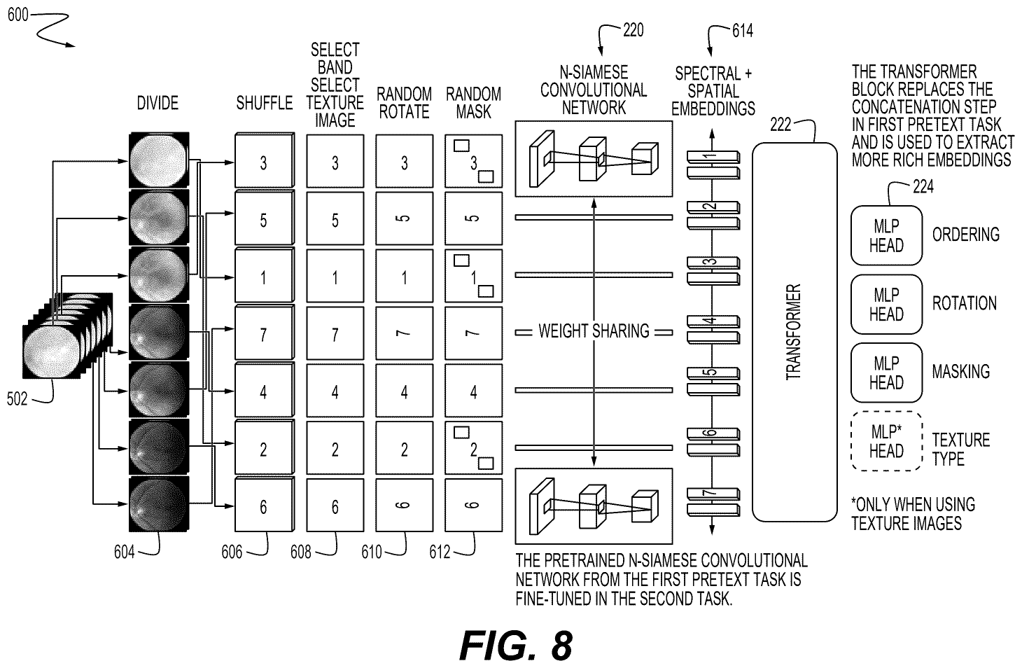

FIG. 8 is an illustration of a second pretext task 600 for training a medical imaging system. In the second pretext task, self-supervised learning is used to construct a spatial-spectral feature extractor. To this end, preprocessing of the same unlabelled multispectral image 502 is altered to introduce a number of other artificial labels related to image modifications. The artificial labels may be the same or different from those applied in the first pretext task. The multispectral image 502 may be split into N groups 604 of images, each image containing light at one of the M wavelengths. As a non-limiting example, the multispectral image 602 may include 91 wavelengths and 7 groups 604 may be formed, each group 604 containing 13 images. In an embodiment, various numbers of images may be placed in each group 604. The positions of the groups 604 may then be shuffled, either randomly or according to a predetermined sequence, forming N shuffled groups 606. One image 608 is selected from each group. The N selected images 608 may be randomly selected among the N shuffled groups 606. Alternatively, each selected image 608 may be a texture image calculated on the basis of the images contained in each shuffled group 606. A geometric transformation, for example a rotation, may selectively be applied to one of more of the N first selected images 608, producing N selected (and transformed) images 610. Finally, an anatomical mask may be selectively applied to the N selected images 610, producing N selected (and masked) images 612.

The N selected (and masked) images 612 are applied for training the convolutional model, for example the N-Siamese convolutional network 220 that outputs a spatial-spectral feature vector 614. The spatial-spectral feature vector 614 is then applied to a sequential information analysis model, for example the transformer encoder 222 configured to perform linear and/or nonlinear transformations defined by weights or parameters that may characterize every neurons in every layers of a neural network. The sequential information analysis model outputs a number of class embeddings. The class embeddings are combined, for example concatenated, and input to the one or more classification heads 224 that output positive or negative indications of the various artificial labels. As expressed in the discussion of FIG. 2, these indications may be compared with the actually applied artificial labels in order to calculate losses and update parameters of the machine learning system 216. In this second pretext task 600, the classification heads may also be specially configured to identity the order, geometric transformation (e.g., rotation), masking and texture type for the N selected images 612.

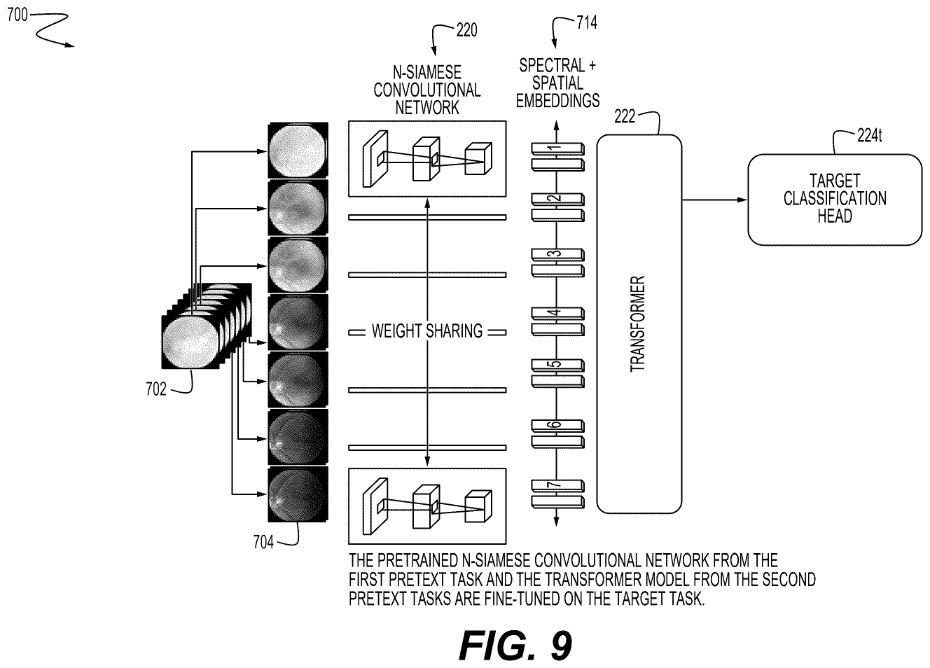

FIG. 9 is an illustration of a target task 700 for training a medical imaging system. In this target task, previously trained N-Siamese convolutional network 220 and transformer encoder 222 are further trained, and a target classification head 224t configured to detect a particular biomarker will also be trained. A multispectral image 702 contains K images are K corresponding wavelengths. The overall spectrum occupied by the K wavelength is the same as, or a subset of, the M wavelengths of the multispectral image used in the first and second pretext task. The multispectral image 702 contains a label indicating whether or not the particular biomarker is present in the biological tissue represented in the multispectral image 702. K images at each of the K respective wavelengths are applied to the convolutional model, for example the N-Siamese convolutional network 220 that outputs a spatial-spectral feature vector 714. The spatial-spectral feature vector 714 is then applied to the sequential information analysis model, for example the transformer encoder 222, which outputs a number of class embeddings. The class embeddings are combined, for example concatenated, and input to the target classification head 224 that output a positive or negative indication of the presence of the biomarker in the biological tissue. This indication may be compared with the label in order to calculate a loss and update parameters of the machine learning system 216.

In a non-limiting example, the first and second pretext tasks may be executed using a few thousands of relatively inexpensive unlabelled images. The target task may be executed using only a few hundreds of labelled images. Overall, the number of labelled images used for training the machine learning system 216 is significantly reduced. The preprocessing applied to unlabelled images in the first and second pretext tasks significantly improves the overall performance and significantly reduces the cost of using the machine learning system 216.

It may be observed that before the first pretext task, the various weights and parameters of the machine learning system 216 may have been randomly initiated. Thereafter, in the course of the learning process, these weights and parameters are progressively updated at various iterations of a forward propagation and backward propagation. The forward propagation consists in feeding the preprocessed images through the machine learning system 216. The backpropagation involves comparing the prediction output by the classification heads 224 with the actual instructions provided by the preprocessing controller 212. The model loss calculated by the loss calculator 216 may initially be large and becomes smaller as the various weights and parameters of the machine learning system 216 are adjusted. At every iteration, several images may be processed in batches (e.g. 16 images in a batch), every batch being processed until all images having been handled at the end of one epoch. This process may be repeated over multiple epochs until the learning process has reached an optimal point where a validation loss is minimized. Once the learning process is completed, a set of weights that are “pre-trained” to be effective at condensing the information and extracting relevant features, has been obtained. Instead of randomly initiating the weights in a subsequent learning process, better results are expected when starting a next phase using these “pre-trained” weights.

It may be observed that the processing system 202 as a whole (FIG. 4) is fundamentally modified by the above-described improvements because it allows to rapidly and economically train the machine learning system 216 by initially using unlabelled images of biological tissues.

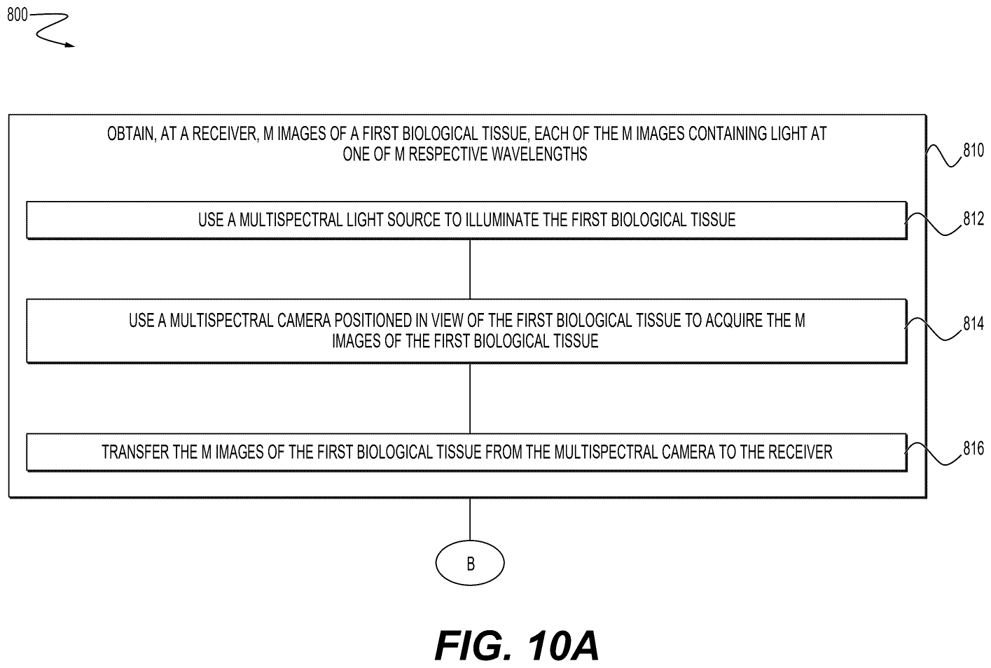

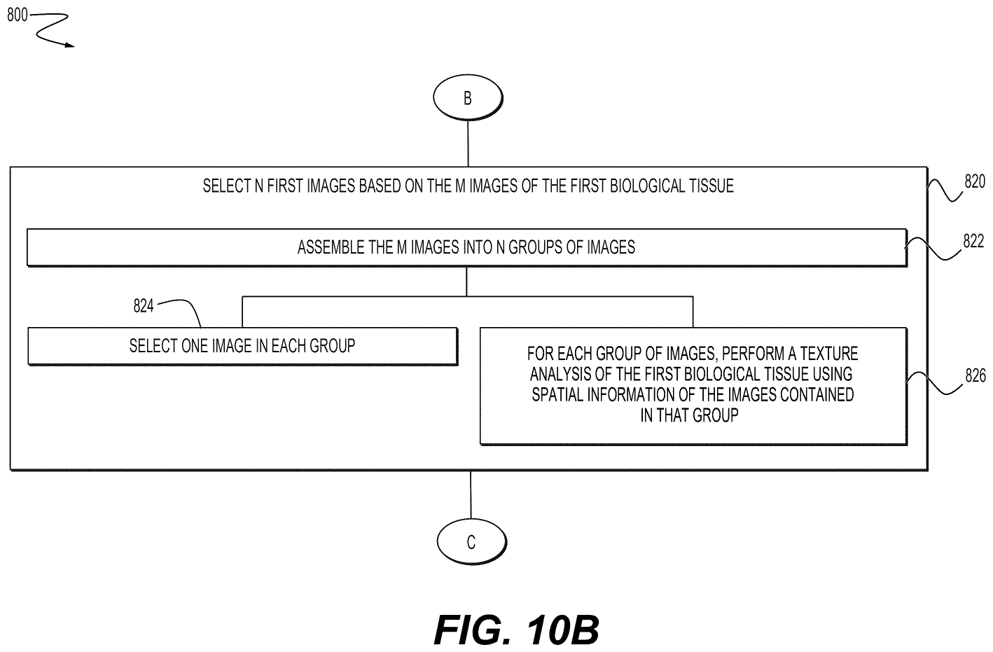

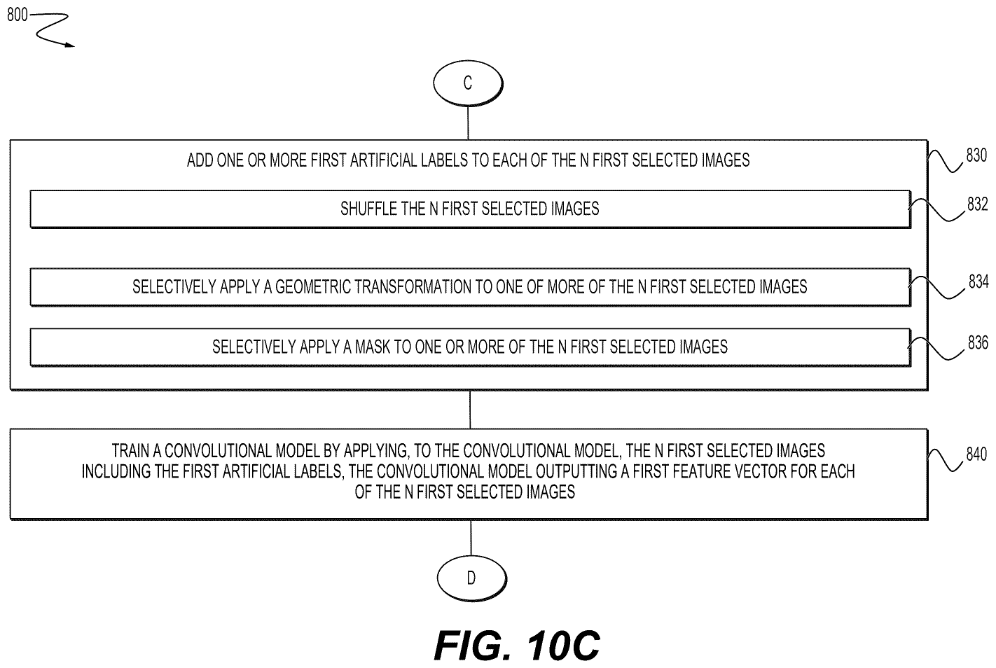

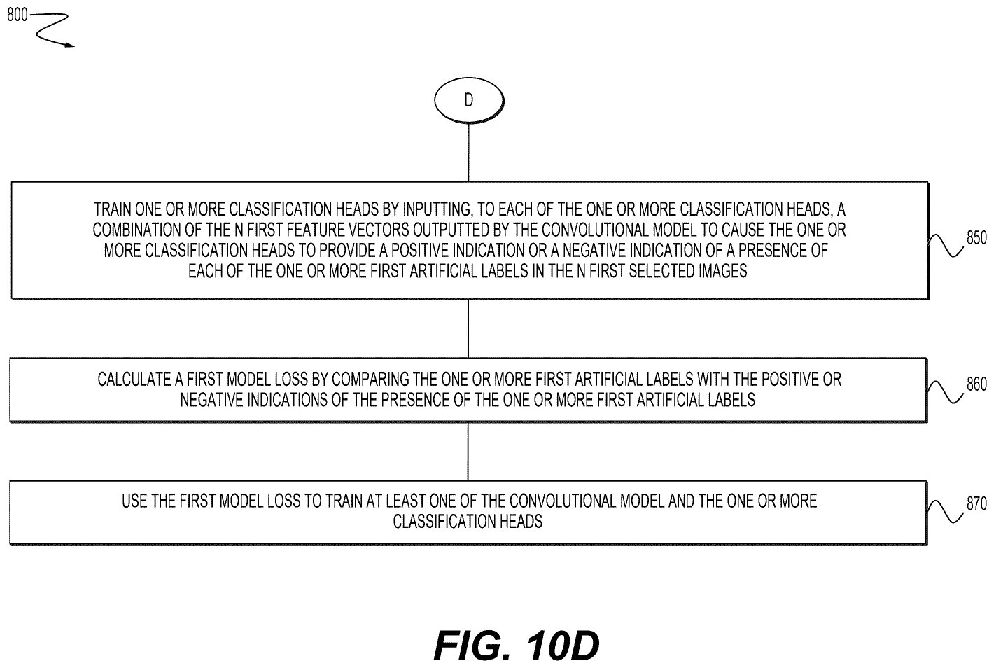

FIGS. 10a to 10d are a sequence diagram showing operations of a first pretext task for training a medical imaging system. On FIGS. 10a to 10d, a sequence 800 comprises a plurality of operations, some of which may be executed in variable order, some of the operations possibly being executed concurrently, some of the operations being optional.

At operation 810, a receiver obtains M images of a first biological tissue. The first biological tissue may, for example and without limitation, be a retina of a subject, a portion of the skin of a subject, or any other anatomical image containing light at two or more wavelengths. Each of the M images contains light at one of M respective wavelengths. In this context, M is an integer number at least equal to 3. Operation 810 may comprise one or more sub-operations 812, 814 and 816. At sub-operation 812, a multispectral light source is used to illuminate the first biological tissue. At sub-operation 814, a multispectral camera positioned in view of the first biological tissue is sued to acquire the M images of the first biological tissue. The M images of the first biological tissue are transferred from the multispectral camera to the receiver at sub-operation 816. Alternatively, the receiver may obtain the M images of the first biological tissue from a network via a communication link, from a computer disk, from a portable memory device, and the like.

N first images are selected based on the M images of the first biological tissue at operation 820. In this context, N is an integer number at least equal to 2 and may be consistent with the size of the N-Siamese convolutional network 220 (FIG. 4). M may be an integer multiple of N. Each of the N groups of images may either contain an equal number of images (i.e., N/M images) or contain an unequal number of images. The N first selected images may simply be a subset of the M images of the first biological tissue, and may possibly be randomly selected among the M images.

In an embodiment, operation 820 may comprise one or more sub-operations 822, 824 and 826. At sub-operation 822, the M images of the first biological tissue are assembled into N groups of images, each group containing consecutive wavelengths. At operation 824, one image may simply be selected in each of the N groups of images. As an alternative to operation 824, operation 826 comprises forming each of the N first selected images as a texture image obtained by performing a texture analysis of the first biological tissue using spatial information of the images contained in a respective one the N groups of images, the texture analysis being resolved over the wavelengths of the images contained in the respective one of the N groups of images.

One or more first artificial labels is added to each of the N first selected images at operation 830. Various types of artificial labels may be added to the N first selected images. For example and without limitation, operation 830 may comprise one or more sub-operations 832, 834 and 836. In one artificial labelling example, the N first selected images may be shuffled (their positions being changed in relation to their natural order among the M wavelengths) at sub-operation 832, If the M images have been assembled in groups at sub-operation 822, the actual N group of images may be shuffled prior to the selected of the N first selected images at operation 824, or before or after the forming of the N texture images. In all cases, the shuffling may be according to a predetermined arrangement or may be randomly performed. In another artificial labelling example, a geometric transformation may selectively be applied to one of more of the N first selected images at sub-operation 834. For example and without limitation, the geometric transformation may comprise an orientation assigned to each of the N first selected images, at least some of the N first selected images being rotated by 90 or −90 degrees, a remainder of the N first selected images being not rotated. Other geometric transformations, for example an isometric transformation, a conformal transformation, or a similarity transformation, are also contemplated. In an embodiment, the geometric transformation may be randomly applied to the one of more of the N first selected images. In yet another artificial labelling example, a mask selected among a plurality of predefined masks may be applied to one or more of the N first selected images at sub-operation 836. The mask may be randomly selected and the one or more of the N first selected images to which the masks are applied may also be randomly selected. These masks may be anatomical masks selected in light of their discriminatory power for a given classification task, for example for classifying the presence of specific biomarkers.

Once at least one first artificial label has been added to each of the N first selected images, a convolutional model is trained at operation 840 by applying, to the convolutional model, the N first selected images including the first artificial labels. This causes the convolutional model to output a first feature vector for each of the N first selected images. The first feature vector outputted by the convolutional model for each of the N first selected images may contain spatial features of the first biological tissue. One or more classification heads are then trained at operation 850 by inputting, to each of the one or more classification heads, a combination, for example a concatenation, of the N first feature vectors outputted by the convolutional model. This causes the one or more classification heads to provide a positive indication or a negative indication of a presence of each of the one or more first artificial labels in the N first selected images.

In more details, the machine learning system 216 calculates a confidence that a label that appears in the N first selected images is of a given class. Where there are only two possible classes (e.g., positive and negative), a predetermined threshold (e.g., 0.5) is applied. A positive or negative indication is provided depending on whether the confidence is above or below the threshold. When there are more than one classes (e.g., left rotation, right rotation, no rotation) the machine learning system 216 outputs a confidence for each possible class and the one that is highest is selected as the classification. For the artificial labels related to shuffling of the N selected images, there may be hundreds or even thousands of possible classes, depending on the value of N. In this case, the machine learning system 216 may predict a probability for each class.

Each of the one or more classification heads may for example be formed as a multi-layer perceptron. Regardless, various types of classification heads may be used depending on the type of artificial labelling applied at operation 830 and at its sub-operations. In one example, a first classification head may provide, for each of the N first selected images, a classification related to a position of that selected image among the M images of the first biological tissue. A second classification head may provide a classification related to the geometric transformation applied to the one or more of the N first selected images. A third classification head may provide, for each of the one or more of the N first selected images, a classification related to a mask having been selectively applied to each of the one or more of the N first selected images. A fourth classification head may provide, for each of the N first selected images, a classification related to a texture type selected from an energy type, a contrast type, a correlation type and a homogeneity type.

At operation 860, a first model loss may be calculated by comparing the one or more first artificial labels with the positive or negative indications of the presence of the one or more first artificial labels. Then at operation 870, the first model loss may be used to train at least one of the convolutional model and the one or more classification heads.

While the sequence 800 has been described in the context of one multispectral image of a first biological tissue containing M images at M corresponding wavelengths, training of the machine learning system 216 and of its components may be performed using hundreds or thousands of images. The M images of the first biological tissue may therefore be included in a first plurality of multispectral images of a first corresponding plurality of biological tissues, the convolutional model and the one or more classification heads being trained using the first plurality of multispectral images using the operations of the sequence 800.

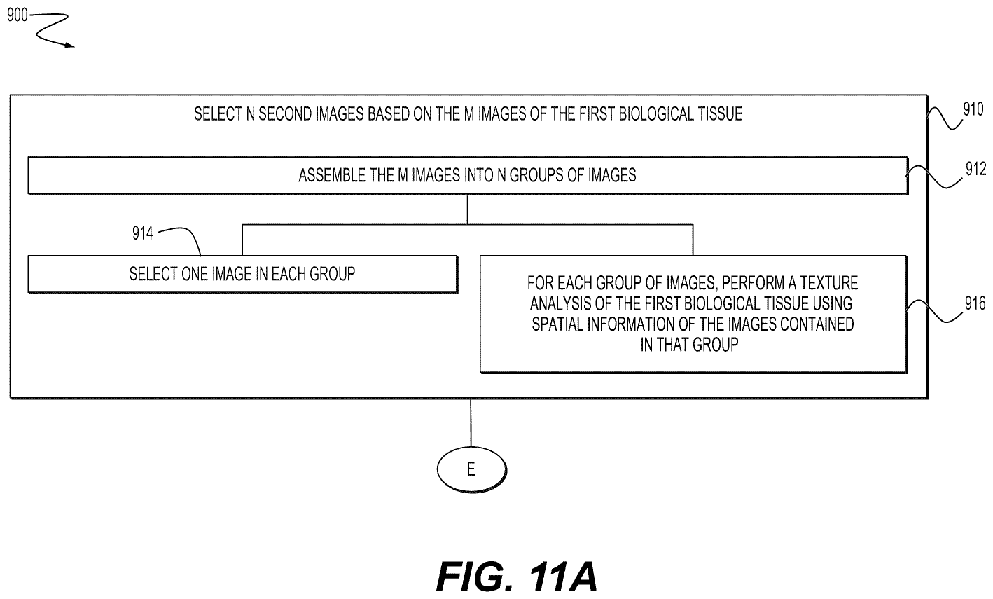

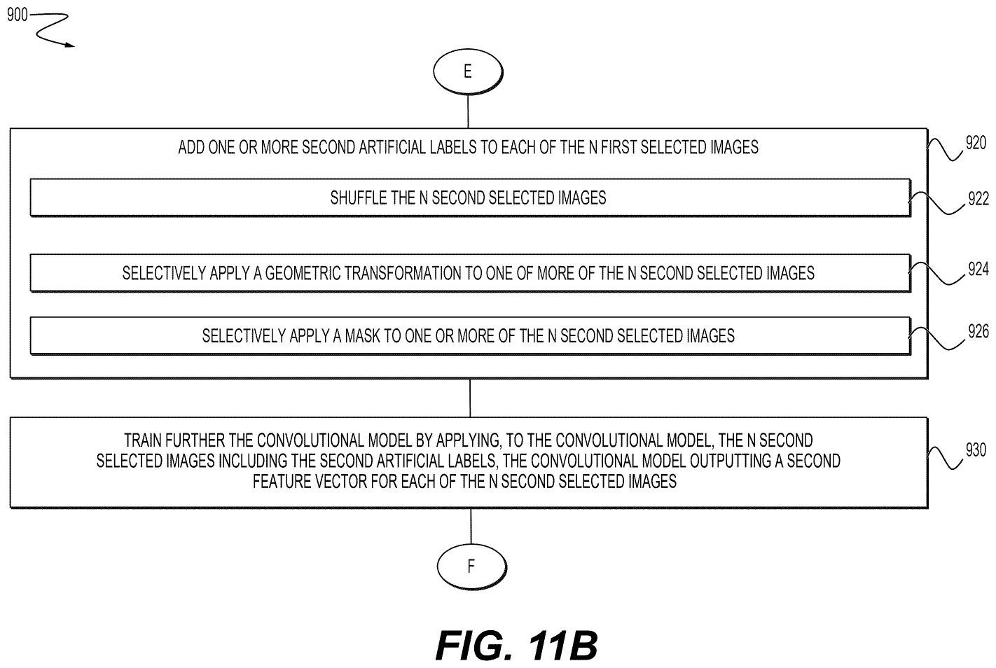

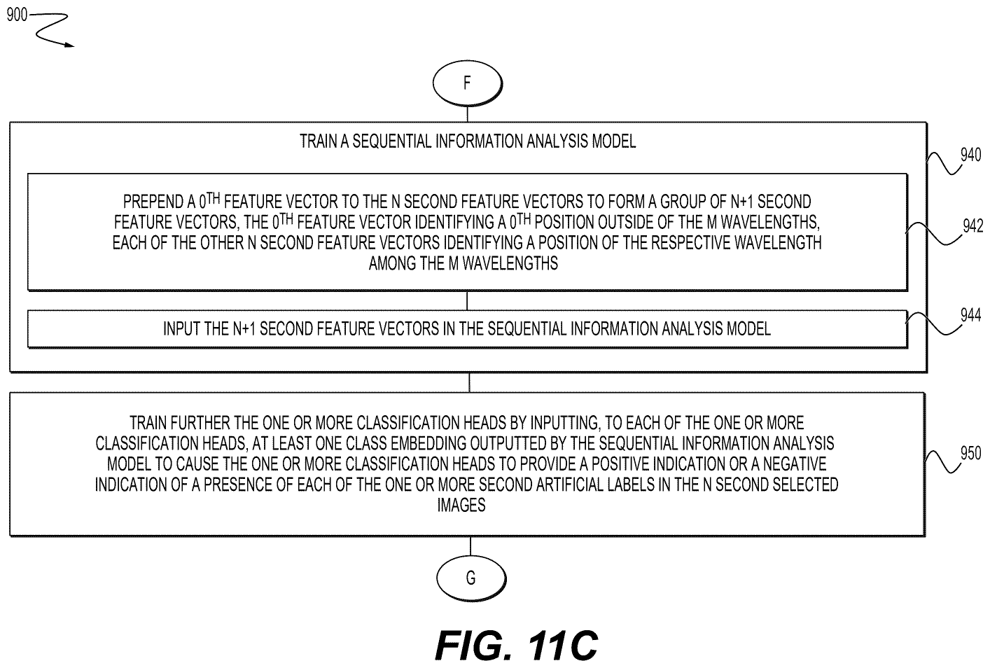

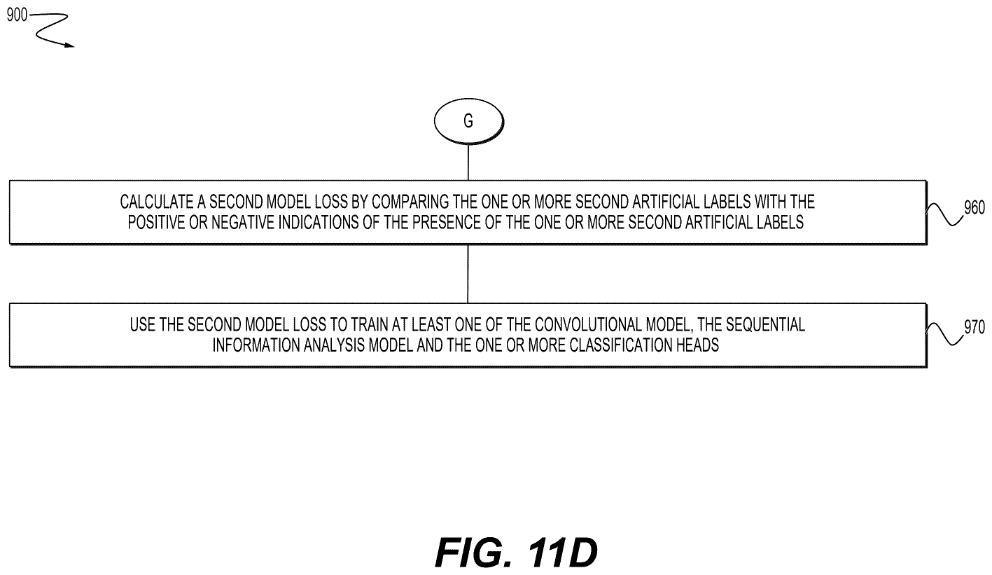

FIGS. 11a to 11d are a sequence diagram showing operations of the second pretext task for training a medical imaging system. On FIGS. 11a to 11d, a sequence 900 comprises a plurality of operations, some of which may be executed in variable order, some of the operations possibly being executed concurrently, some of the operations being optional.

At operation 910, N second images are selected among the M images of the first biological tissue. It may be noted that the M images of the first biological tissue are the same as those obtained at operation 810 of the first pretext task (FIG. 10a). The N second selected images are not necessarily the same as the N first images selected at operation 820 of the first pretext task (FIG. 10b). Also, the number N of second selected images is not necessarily equal to the number N of first selected images, inasmuch as N remains an integer number at least equal to 2. In an embodiment, operation 910 may comprise one or more sub-operations 912, 914 and 916. At sub-operation 912, the M images of the first biological tissue are assembled into N groups of images, each group containing consecutive wavelengths. At operation 914, one image may simply be selected in each of the N groups of images. As an alternative to operation 914, operation 916 comprises forming each of the N second selected images as a texture image obtained by performing a texture analysis of the first biological tissue using spatial information of the images contained in a respective one the N groups of images, the texture analysis being resolved over the wavelengths of the images contained in the respective one of the N groups of images.

One or more second artificial labels is added to one or more of the N second selected images at operation 920. Although the second artificial labels are not necessarily the same as the first artificial labels added to the N first selected images at operation 830 (FIG. 10c), the same or equivalent techniques may be employed in operation 920. Namely, operation 920 may comprise one or more sub-operations 922, 924 and 926 that are equivalent to sub-operations 832, 834 and 836.

The convolutional model is trained further at operation 930 by applying, to the convolutional model, the N second selected images including the one or more second artificial labels, the convolutional model outputting a second feature vector for each of the N second selected images. The second feature vector outputted by the convolutional model for each of the N second selected images may contain spatial-spectral features of the first biological tissue.

A sequential information analysis model is then trained at operation 940. The sequential information analysis model may for example be transformer encoder, a long short-term memory model or a recurrent neural network. Operation 940 may comprise one or more sub-operations 942 and 944. At sub-operation 942, a 0^{th} feature vector is prepended to the N second feature vectors to form a group of N+1 second feature vectors, the 0^{th} feature vector identifying a 0^{th} position outside of the M wavelengths, each of the other N second feature vectors identifying a position of the respective wavelength among the M wavelengths. At sub-operation 944, the N+1 second feature vectors is input in the sequential information analysis model.

Then at operation 950, the one or more classification heads are trained further by inputting, to each of the one or more classification heads, at least one class embedding outputted by the sequential information analysis model to cause the one or more classification heads to provide a positive indication or a negative indication of a presence of each of the one or more second artificial labels in the N second selected images. Generally speaking, the above description of possible manners in which the machine learning system 216 determines confidence values for the various labels in the course of the first pretext task also applies in the second pretext task.

At operation 960, a second model loss may be calculated by comparing the one or more second artificial labels with the positive or negative indications of the presence of the one or more second artificial labels. Then at operation 970, the second model loss may be used to train at least one of the convolutional model, the sequential information analysis model and the one or more classification heads.

As in the case of the sequence 800, the sequence 900 has been described in the context of one multispectral image of a first biological tissue containing M images at M corresponding wavelengths, training of the machine learning system 216 and of its components may be performed using hundreds or thousands of images. The M images of the first biological tissue may therefore be included in a first plurality of multispectral images of a first corresponding plurality of biological tissues, the convolutional model, the sequential information analysis model and the one or more classification heads being trained using the first plurality of multispectral images using the operations of the sequence 900.

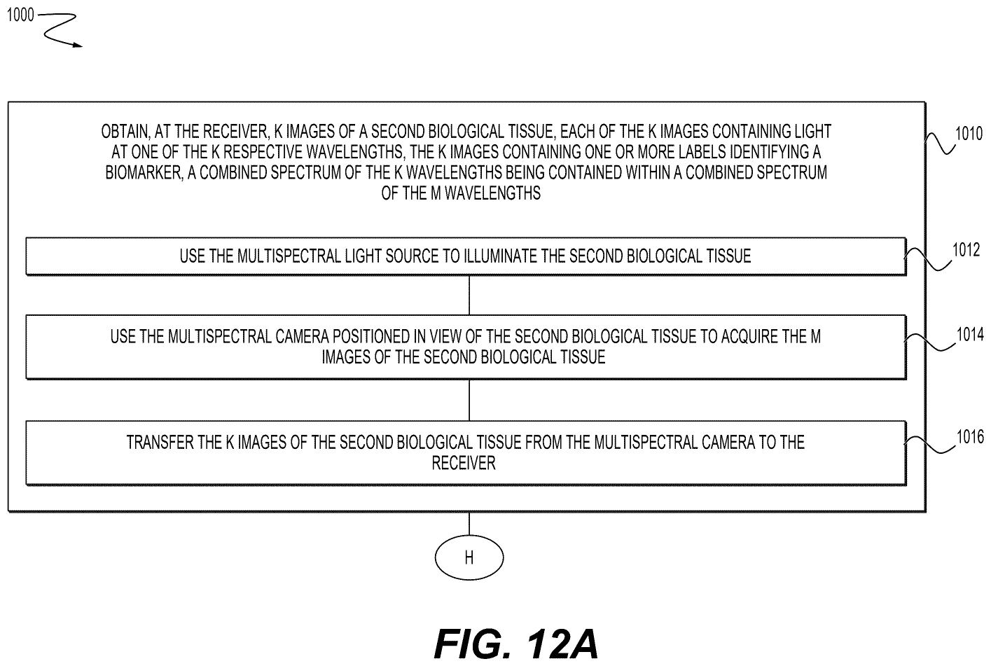

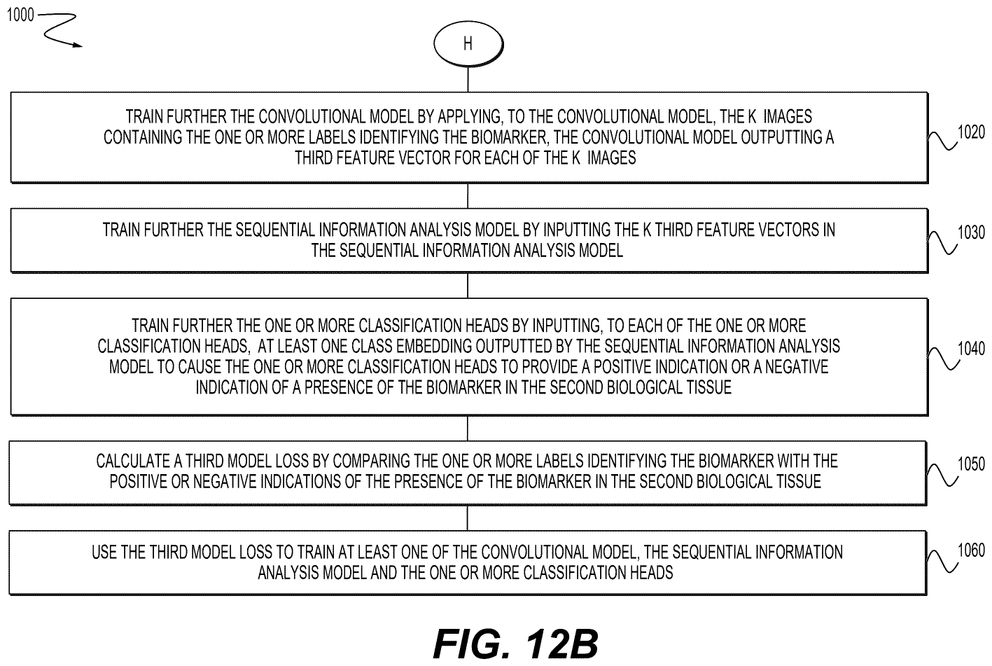

FIGS. 12a and 12b are a sequence diagram showing operations of the target task for training a medical imaging system. On FIGS. 12a and 12b, a sequence 1000 comprises a plurality of operations, some of which may be executed in variable order, some of the operations possibly being executed concurrently, some of the operations being optional.

At operation 1010, a receiver obtains K images of a second biological tissue. The second biological tissue may also be, for example and without limitation, be retina of a subject, a portion of the skin of a subject, or any other anatomical image containing light at two or wavelengths. Each of the K images contains light at one of K respective wavelengths. The K images form a multispectral image of the second biological tissue, the multispectral image including one or more labels indicative of the presence or absence of a particular biomarker. A value of K may the same as, or differ from, a value of M. The overall spectrum occupied by the K wavelength may be the same as, or a subset of, the M wavelengths of the multispectral image used in the first and second pretext task. In this manner, the machine learning system 216 having been trained over the spectrum covered by the M wavelengths is well prepared for operating over the spectrum covered by the K wavelengths. Regardless, k also is an integer number at least equal to 3. Operation 1010 may comprise one or more sub-operations 1012, 1014 and 1016. At sub-operation 1012, a multispectral light source is used to illuminate the second biological tissue. At sub-operation 1014, a multispectral camera positioned in view of the second biological tissue is sued to acquire the K images of the second biological tissue. The K images of the second biological tissue are transferred from the multispectral camera to the receiver at sub-operation 1016. Alternatively, the receiver may obtain the K images of the second biological tissue from a network via a communication link, from a computer disk, from a portable memory device, and the like.

The convolutional model is trained further at operation 1020 by applying, to the convolutional model, the K images containing the one or more labels identifying the particular biomarker, the convolutional model outputting a third feature vector for each of the K images. The sequential information analysis model is also trained further at operation 1030 by inputting the K third feature vectors in the sequential information analysis model. Then, at operation 1040, the one or more classification heads are trained further by inputting, to each of the one or more classification heads, at least one class embedding outputted by the sequential information analysis model to cause the one or more classification heads to provide a positive indication or a negative indication of a presence of the particular biomarker in the second biological tissue.

In an embodiment, the one or more classification heads may comprise a target classification head configured to provide the positive indication or negative indication of the presence of the particular biomarker in the second biological tissue.

At operation 1050, a third model loss may be calculated by comparing the one or more labels identifying the particular biomarker with the positive or negative indications of the presence of the particular biomarker in the second biological tissue. Using the third model loss, at least one of the convolutional model, the sequential information analysis model and the one or more classification heads may be trained at operation 1060.

The sequence 1000 has been described in the context of one multispectral image of a second biological tissue containing K images at MK corresponding wavelengths. In fact, training of the machine learning system 216 and of its components may be performed using hundreds or thousands of images. The K images of the second biological tissue may therefore be included in a second plurality of labelled multispectral images of a second corresponding plurality of biological tissues, the convolutional model, the sequential information analysis model and the one or more classification heads being trained further using the second plurality of multispectral images using the operations of the sequence 1000.

Once the medical imaging system 200 has been sufficiently trained, as may for example be determined by a medical practitioner evaluation its classification performance, unlabelled images of biological tissues may be applied in a clinical context to the medical imaging system 200, with or without further training of the machine learning system 216, in order to help in the detection of the presence of particular biomarkers in actual subjects.

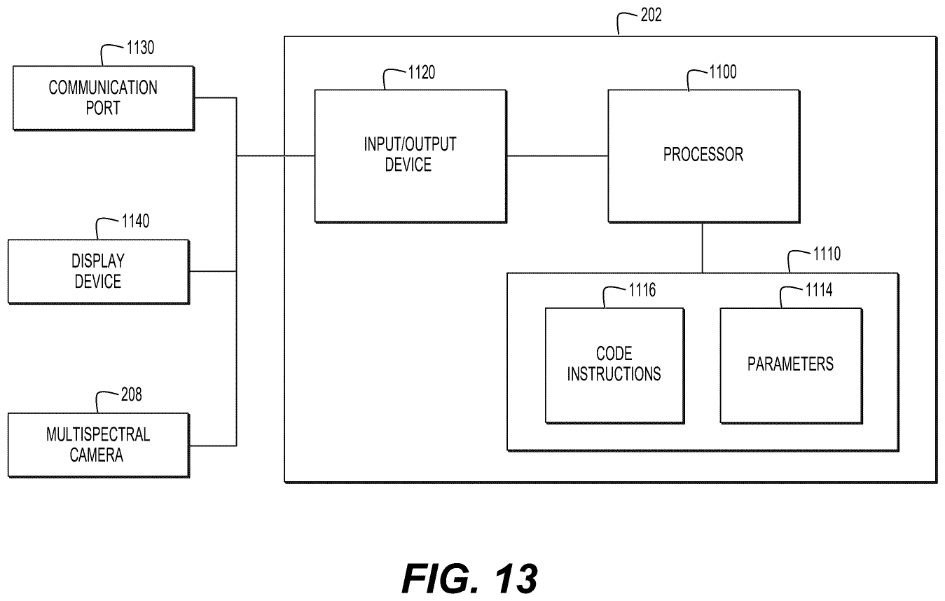

Each of the operations of the sequences 400, 800, 900 and 1000 may be configured to be processed by one or more processors, the one or more processors being coupled to a memory device. For example, FIG. 13 is a block diagram of the processing system 202 part of the medical imaging system 200. On FIG. 13, the processing system 202 includes a processor or a plurality of cooperating processors, represented as a processor 1100 for simplicity. In a practical implementation, it is expected that the processor 1100 will comprise a plurality of processors, some of which will implement the neurons of a neural network forming the machine learning system 216. The processing system also comprises, a memory device or a plurality of memory devices (represented as a memory device 1110 for simplicity), an input/output device or a plurality of input/output devices (represented as an input/output device 1120). Distinct input and output devices are also contemplated. The processor 1100 is operatively connected to the memory device 1110 and to the input/output device 1120. The memory device 1110 stores a list of parameters 1114, including for example the various parameters and weights of the machine learning system 216. The memory device 1110 may comprise a non-transitory computer-readable media for storing code instructions 1116 that are executable by the processor 1100 to execute the operations of the sequences 400, 800, 900 and/or 1000.

The image receiver 204 (FIG. 4) may be implemented within the input/output device 1120. The input/output device 1120 may be communicatively coupled to any one or more of the multispectral camera 208, a communication port 1130 for receiving multispectral image and/or for transmitting classification results, a display device 1140 for showing classification results, and the like.

While the present technology has been described in the context of detecting biomarkers in biological tissues, the present technology is readily adaptable to the detection of artefacts in various types multispectral images. In a non-limiting example, the detection of ore deposits in multispectral geomatic images, possibly obtained by satellites, is also contemplated. In an example, a method for detecting artefacts in a multispectral image may comprise obtaining, at a receiver, M images contained in the multispectral image, each of the M images containing light at one of M respective wavelengths, applying, to each of the M images, L masks for obtaining M×L pixel groups, applying K statistical calculations to each of the M×L pixel groups, assembling results of the statistical calculations in M feature vectors, each feature vector containing L×K results for a corresponding one of the M wavelengths, and using a machine learning system to extract, from the M feature vectors, a positive indication or a negative indication of a presence of a given artefact in the multispectral image. In another example, a method for training multispectral imaging system may comprise obtaining, at a receiver, M images contained in a multispectral image, each of the M images containing light at one of M respective wavelengths, selecting N first images based on the M images, adding one or more first artificial labels to each of the N first selected images, training a convolutional model by applying, to the convolutional model, the N first selected images including the first artificial labels, the convolutional model outputting a first feature vector for each of the N first selected images, and training one or more classification heads by inputting, to each of the one or more classification heads, a combination of the N first feature vectors outputted by the convolutional model to cause the one or more classification heads to provide a positive indication or a negative indication of a presence of each of the one or more first artificial labels in the N first selected images. A system comprising a receiver and a processor (e.g., a group of processors, possibly including a neural network or another machine learning system) may execute any one of both of these methods.

While the above-described implementations have been described and shown with reference to particular operations performed in a particular order, it will be understood that these operations may be combined, sub-divided, or re-ordered without departing from the teachings of the present technology. At least some of the operations may be executed in parallel or in series. Accordingly, the order and grouping of the operations is not a limitation of the present technology.

It should be expressly understood that not all technical effects mentioned herein need to be enjoyed in each and every embodiment of the present technology.

Modifications and improvements to the above-described implementations of the present technology may become apparent to those skilled in the art. The foregoing description is intended to be exemplary rather than limiting. The scope of the present technology is therefore intended to be limited solely by the scope of the appended claims.

1. A method for detecting biomarkers in a biological tissue, the method comprising:
obtaining, at a receiver, M images of the biological tissue, each of the M images containing light at one of M respective wavelengths;
applying, to each of the M images, L masks for obtaining M×L pixel groups;
applying K statistical calculations to each of the M×L pixel groups;
assembling results of the statistical calculations in M feature vectors, each feature vector containing L×K results for a corresponding one of the M wavelengths; and
using a machine learning system to extract, from the M feature vectors, a positive indication or a negative indication of a presence of a given biomarker in the biological tissue.

2. The method of claim 1, wherein the M images of the biological tissue are images of a retina of a subject.

3. The method of claim 1, wherein:
M is an integer number at least equal to 2;
L is an integer number at least equal to 1; and
K is an integer number at least equal to 1.

4. The method of claim 1, wherein each of the L masks is an anatomical mask, a combination of each mask with each statistical calculation having a discriminatory power for classifying a presence of a specific biomarker in the biological tissue.

5. The method of claim 4, wherein each anatomical mask is configured to highlight, in the M images of the biological tissue, a corresponding pixel group defining an element selected from an optic nerve hypoplasia, a blood vessel, an optic nerve head, a vessel inside the optic nerve head, a contour of a blood vessel, pigment spots, a drusen, and a retinal background.

6. The method of claim 1, wherein each of the K statistical calculation is selected from an average, a variance, a skewness, a kurtosis, a standard deviation, a median, a smallest value, a largest value, a first, second or third quartile, and any combination thereof.

7. The method of claim 1, wherein the machine learning system extracts the positive or negative indication of the presence of the given biomarker in the biological tissue from the M feature vectors by:
prepending a 0^{th} feature vector to the M feature vectors to form a group of M+1 feature vectors, the 0^{th} feature vector identifying a 0^{th} position outside of the M wavelengths, each of the other M feature vectors identifying a position of the respective wavelength among the M wavelengths;
inputting the M+1 feature vectors in a sequential information analysis model;
outputting at least one class embedding from the sequential information analysis model; and
applying the at least one class embedding to a classification head, the classification head outputting the positive or negative indication of the presence of the given biomarker in the biological tissue.

8. The method of claim 7, wherein the sequential information analysis model is selected from a transformer encoder, a long short-term memory model and a recurrent neural network.

9. The method of claim 7, wherein the at least one class embedding applied to the classification head is a 0^{th} class embedding of M+1 class embeddings outputted from the sequential information analysis model.

10. The method of claim 7, wherein the classification head is a multi-layer perceptron.

11. The method of claim 7, wherein the classification head comprises zero or more fully connected hidden layers and a fully connected classification layer.

12. The method of claim 1, further comprising:
using a multispectral light source to illuminate the biological tissue;
using a multispectral camera positioned in view of the biological tissue to acquire the M images of the biological tissue; and
transferring the M images of the biological tissue from the multispectral camera to the receiver.

13. (canceled)

14. A medical imaging system for detecting biomarkers in a biological tissue, the medical imaging system comprising:
a receiver configured to obtain M images of the biological tissue, each of the M images containing light at one of M respective wavelengths; and
a processor operatively connected to the receiver, the processor being configured to:
apply, to each of the M images, L masks for obtaining M×L pixel groups,
apply K statistical calculations to each of the M×L pixel groups,
assemble results of the statistical calculations in M feature vectors, each feature vector containing L×K results for a corresponding one of the M wavelengths, and
use a machine learning system to extract, from the M feature vectors, a positive indication or a negative indication of a presence of a given biomarker in the biological tissue.

15.-25 (canceled)

26. The system of claim 14, wherein
the receiver is further configured to obtain a plurality of groups of images, each group of images corresponding to one of a plurality of biological tissues, each group of images including M respective images containing light the M respective wavelengths;
the processor is further configured to:
for each group of images, calculate a respective model loss by comparing a respective label contained in the group of images with a respective positive or negative indication of the presence of the given biomarker in the corresponding biological tissue, and
use the calculated model loss to train the machine learning system.

27. (canceled)

28. (canceled)

29. A method for training a medical imaging system, the method comprising:
obtaining, at a receiver, M images of a first biological tissue, each of the M images containing light at one of M respective wavelengths;
selecting N first images based on the M images of the first biological tissue;
adding one or more first artificial labels to each of the N first selected images;
training a convolutional model by applying, to the convolutional model, the N first selected images including the first artificial labels, the convolutional model outputting a first feature vector for each of the N first selected images; and
training one or more classification heads by inputting, to each of the one or more classification heads, a combination of the N first feature vectors outputted by the convolutional model to cause the one or more classification heads to provide a positive indication or a negative indication of a presence of each of the one or more first artificial labels in the N first selected images.

30. The method of claim 29, further comprising:
calculating a first model loss by comparing the one or more first artificial labels with the positive or negative indications of the presence of the one or more first artificial labels; and
using the first model loss to train at least one of the convolutional model and the one or more classification heads.

31. The method of claim 29, wherein each of the one or more classification heads is a multi-layer perceptron.

32. The method of claim 29, wherein the N first selected images are a subset of the M images of the first biological tissue.

33. The method of claim 29, further comprising:
assembling the M images of the first biological tissue into N groups of images, each group containing consecutive wavelengths; and
selecting the N first selected images by selecting one image based on each of the N groups of images.

34. The method of claim 33, wherein each of the N groups of images contains an equal number of images.

35.-102. (canceled)
